(19) United States
(12) Patent Application Publication
(10) Pub. No.: US 2006/0149062 A1
(43) Pub. Date: Jul. 6, 2006

(54) Sulfanyl substituted phenyl methanones

(76) Inventors: Synese Jolidon, Blauen (CH); Robert Narquizian, St. Louis (FR); Roger David Norcross, Olsberg (CH); Emmanuel Pinard, Linsdorf (FR)

Correspondence Address: HOFFMANN-LA ROCHE INC.;PATENT LAW DEPARTMENT, 340 KINGSLAND STREET, NUTLEY, NJ, 07110, US

(21) Appl. No.: 11/324,991

(22) Filed: Jan. 3, 2006

(30) Foreign Application Priority Data
Jan. 6, 2005 (EP) 05100066.9

(51) Int. Cl. C07D 403/04; C07D 241/04

(52) U.S. Cl. 544/295; 544/360; 544/386

(57) ABSTRACT

The present invention relates to compounds of formula I 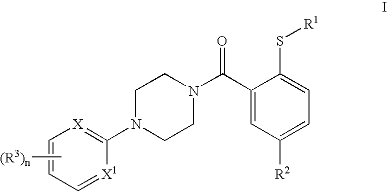 wherein
R¹, R², R³, X, X¹, and n are as defined in the specification and pharmaceutically acceptable acid addition salts thereof. These compounds are good inhibitors of the glycine transporter 1 (GlyT-1) for the treatment CNS disorders, such as schizophrenia, cognitive impairment, and Alzheimer's disease.

### PRIORITY DATA

This application claims the benefit of European Application No. 05100066.9, filed Jan. 6, 2005, which is hereby incorporated by reference in its entirety.

### BACKGROUND OF THE INVENTION

Schizophrenia is a progressive and devastating neurological disease characterized by episodic positive symptoms such as delusions, hallucinations, thought disorders and psychosis and persistent negative symptoms such as flattened affect, impaired attention and social withdrawal, and cognitive impairments (Lewis D A and Lieberman J A, Neuron, 28:325-33, 2000). For decades research has focused on the “dopaminergic hyperactivity” hypothesis which has led to therapeutic interventions involving blockade of the dopaminergic system (Vandenberg R J and Aubrey K R., Exp. Opin. Ther. Targets, 5(4): 507-518, 2001; Nakazato A and Okuyama S, et al., Exp. Opin. Ther. Patents, 10(1): 75-98, 2000). This pharmacological approach poorly address negative and cognitive symptoms which are the best predictors of functional outcome (Sharma T., Br. J. Psychiatry, 174(suppl. 28): 44-51, 1999).

A complementary model of schizophrenia was proposed in the mid-1960s based upon the psychotomimetic action caused by the blockade of the glutamate system by compounds like phencyclidine (PCP) and related agents (ketamine) which are non-competitive NMDA receptor antagonists. Interestingly, in healthy volunteers, PCP-induced psychotomimetic action incorporates positive and negative symptoms as well as cognitive dysfunction, thus closely resembling schizophrenia in patients (Javitt D C et al., Biol. Psychiatry, 45: 668-679, 1999). Furthermore transgenic mice expressing reduced levels of the NMDAR1 subunit display behavioral abnormalities similar to those observed in pharmacologically induced models of schizophrenia, supporting a model in which reduced NMDA receptor activity results in schizophrenia-like behavior (Mohn A R et al., Cell, 98: 427-236, 1999).

Glutamate neurotransmission, in particular NMDA receptor activity, plays a critical role in synaptic plasticity, learning and memory, such that NMDA receptors appear to serve as a graded switch for gating the threshold of synaptic plasticity and memory formation (Wiley, N Y; Bliss T V and Collingridge G L, Nature, 361: 31-39, 1993). Transgenic mice overexpressing the NMDA NR2B subunit exhibit enhanced synaptic plasticity and superior ability in learning and memory (Tang J P et al., Natur, 401-63-69, 1999).

Thus, if a glutamate deficit is implicate in the pathophysiology of schizophrenia, enhancing glutamate transmission, in particular via NMDA receptor activation, would be predicted to produce both anti-psychotic and cognitive enhancing effects.

The amino acid glycine is known to have at least two important functions in the CNS. It acts as an inhibitory amino acid, binding to strychnine sensitive glycine receptors, and it also influences excitatory activity, acting as an essential co-agonist with glutamate for N-methyl-D-aspartate (NMDA) receptor function. While glutamate is released in an activity-dependent manner from synaptic terminals, glycine is apparently present at a more constant level and seems to modulate/control the receptor for its response to glutamate.

One of the most effective ways to control synaptic concentrations of neurotransmitter is to influence their re-uptake at the synapses. Neurotransmitter transporters act by removing neurotransmitters from the extracellular space, and can control their extracellular lifetime and thereby modulate the magnitude of the synaptic transmission (Gainetdinov R R et al, Trends in Pharm. Sci., 23(8): 367-373, 2002).

Glycine transporters, which form part of the sodium and chloride family of neurotransmitter transporters, play an important role in the termination of post-synaptic glycinergic actions and maintenance of low extracellular glycine concentration by re-uptake of glycine into presynaptic nerve terminals and surrounding fine glial processes.

Two distinct glycine transporter genes have been cloned (GlyT-1 and GlyT-2) from mammalian brain, which give rise to two transporters with ˜50% amino acid sequence homology. GlyT-1 presents four isoforms arising from alternative splicing and alternative promoter usage (1a, 1b, 1c and 1d). Only two of these isoforms have been found in rodent brain (GlyT-1a and GlyT-1b). GlyT-2 also presents some degree of heterogeneity. Two GlyT-2 isoforms (2a and 2b) have been identified in rodent brains. GlyT-1 is known to be located in CNS and in peripheral tissues, whereas GlyT-2 is specific to the CNS. GlyT-1 has a predominantly glial distribution and is found not only in areas corresponding to strychnine sensitive glycine receptors but also outside these areas, where it has been postulated to be involved in modulation of NMDA receptor function (Lopez-Corcuera B et al., Mol. Mem. Biol., 18:13-20, 2001). Thus, one strategy to enhance NMDA receptor activity is to elevate the glycine concentration in the local microenvironment of synaptic NMDA receptors by inhibition of GlyT-1 transporter (Bergereon R. et al., Proc. Natl. Acad. Sci. USA, 95:15730-15734, 1998; Chen L. et al., J. Neurophysiol., 89(2): 691-703, 2003).

Glycine transporter inhibitors are suitable for the treatment of neurological and neuropsychiatric disorders. The majority of diseases states implicated are psychoses, schizophrenia (Armer R E and Miller D J, Exp. Opin. Ther. Patents, 11 (4): 563-572, 2001), psychotic mood disorders such as severe major depressive disorder, mood disorders associated with psychotic disorders such as acute mania or depression, associated with bipolar disorders and mood disorders, associated with schizophrenia, (Pralong E T et al., Prog. Neurobiol., 67: 173-202, 2002), autistic disorders (Carlsson M L, J. Neural Trans., 105: 525-535, 1998), cognitive disorders such as dementias, including age related dementia and senile dementia of the Alzheimer type, memory disorders in a mammal, including a human, attention deficit disorders and pain (Armer R E and Miller D J, Exp. Opin. Ther. Patents, 11 (4): 563-572, 2001).

Thus, increasing activation of NMDA receptors via GlyT-1 inhibition can lead to agents that treat psychosis, schizophrenia, dementia and other diseases in which cognitive processes are impaired, such as attention deficit disorders or Alzheimer's disease.

### SUMMARY OF THE INVENTION

The present invention provides compounds of formula I 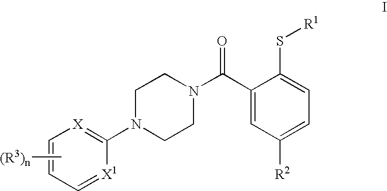
wherein
R¹ is lower alkyl or lower alkyl substituted by halogen;
R² is —S(O)₂-lower alkyl, —S(O)₂NH-lower alkyl, NO₂, or CN;
R³ is halogen, CN, lower alkyl, lower alkyl substituted by halogen, NO₂, —C(O)-lower alkyl or S(O)₂-lower alkyl;
X and X¹ are each independently CR⁴ or N;
R⁴ is hydrogen or halogen; and
n is 0, 1 or 2;
   and pharmaceutically acceptable acid addition salts thereof.

The invention includes all racemic mixtures, all their corresponding enantiomers and/or optical isomers. The present invention also provides pharmaceutical compositions containing compounds of the invention and a pharmaceutically acceptable carrier. The invention also provides methods for manufacturing compounds of the invention and compositions containing them.

Compounds of formula I are good inhibitors of the glycine transporter 1 (GlyT-1), and have a good selectivity to glycine transporter 2 (GlyT-2) inhibitors. The present invention further provides methods for the treatment of diseases related to activation of NMDA receptors via Glyt-1 inhibition, such as psychoses, dysfunction in memory and learning, schizophrenia, dementia and other diseases in which cognitive processes are impaired, such as attention deficit disorders or Alzheimer's disease. The preferred indications using the compounds of the present invention are schizophrenia, cognitive impairment and Alzheimer's disease.

### DETAILED DESCRIPTION OF THE INVENTION

The following definitions of the general terms used in the present description apply irrespective of whether the terms in question appear alone or in combination. It must be noted that, as used in the specification and the appended claims, the singular forms “a”, “an,” and “the” include plural forms unless the context clearly dictates otherwise.

As used herein, the term “alkyl” denotes a saturated straight- or branched-carbon chain containing from 1 to 6 carbon atoms, for example, methyl, ethyl, propyl, isopropyl, n-butyl, i-butyl, 2-butyl, t-butyl and the like. Preferred alkyl groups are groups with 1-4 carbon atoms.

The term “alkyl substituted by halogen” denotes an alkyl group as defined above, wherein at least one hydrogen atom is replaced by halogen, for example the following groups: CF₃, CHF₂, CH₂F, CH₂CF₃, CH₂CHF₂, CH₂CH₂F, CH₂CH₂CF₃, CH₂CH₂CH₂CF₃, CH₂CH₂Cl, CH₂CF₂CF₃, CH₂CF₂CHF₂, CF₂CHFCF₃, C(CH₃)₂CF₃, CH(CH₃)CF₃ or CH(CH₂F)CH₂F. Preferred are CH₂CF₃ or CF₃.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

The present invention provides compounds of formula I 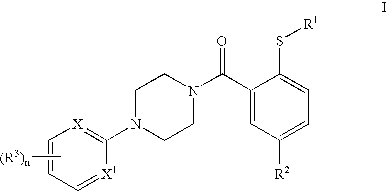
wherein
R¹ is lower alkyl or lower alkyl substituted by halogen;
R² is —S(O)₂-lower alkyl, —S(O)₂NH-lower alkyl, NO₂, or CN;
R³ is halogen, CN, lower alkyl, lower alkyl substituted by halogen, NO₂, —C(O)-lower alkyl or S(O)₂-lower alkyl;
X and X¹ are each independently CR⁴ or N;
R⁴ is hydrogen or halogen; and
n is 0, 1 or 2;
   and pharmaceutically acceptable acid addition salts thereof.

In particular, the present invention provides compounds of formulae IA, IB, and IC: 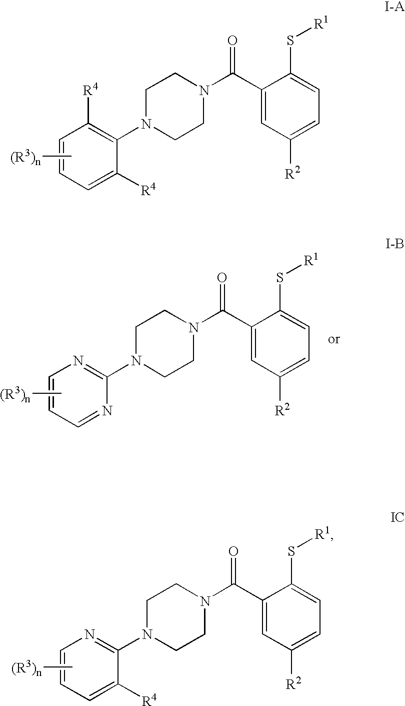
wherein
R¹ is lower alkyl or lower alkyl substituted by halogen;
R² is —S(O)₂-lower alkyl, —S(O)₂NH-lower alkyl, NO₂, or CN;
R³ is halogen, CN, lower alkyl, lower alkyl substituted by halogen, NO₂, —C(O)-lower alkyl or S(O)₂-lower alkyl;
R⁴ is hydrogen or halogen; and
n is 0, 1, or 2;
   and pharmaceutically acceptable acid addition salts thereof.

Preferred compounds of the present application are compounds of formula I, wherein R¹ is lower alkyl. Such compounds are
[4-(3-fluoro-5-trifluoromethyl-pyridin-2-yl)-piperazin-1-yl]-(2-isopropylsulfanyl-5-methanesulfonyl-phenyl)-methanone,
[4-(2-fluoro-4-methanesulfonyl-phenyl)-piperazin-1-yl]-(2-isopropylsulfanyl-5-methanesulfonyl-phenyl)-methanone,
(2-ethylsulfanyl-5-methanesulfonyl-phenyl)-[4-(3-fluoro-5-trifluoromethyl-pyridin-2-yl)-piperazin-1-yl]-methanone,
[4-(2-fluoro-4-methanesulfonyl-phenyl)-piperazin-1-yl]-(2-isobutylsulfanyl-5-methanesulfonyl-phenyl)-methanone,
1-{3-fluoro-4-[4-(2-isopropylsulfanyl-5-methanesulfonyl-benzoyl)-piperazin-1-yl]-phenyl}-ethanone,
[4-(2-chloro-4-nitro-phenyl)-piperazin-1-yl]-(2-isopropylsulfanyl-5-methanesulfonyl-phenyl)-methanone,
4-isopropylsulfanyl-N-methyl-3-[4-(4-trifluoromethyl-phenyl)-piperazine-1-carbonyl]-benzenesulfonamide and
(2-isopropylsulfanyl-5-nitro-phenyl)-[4-(4-trifluoromethyl-phenyl)-piperazin-1-yl]-methanone.

Preferred compounds of the present application are further compounds of formula I, wherein R¹ is lower alkyl substituted by halogen. Such compounds are
[4-(3-fluoro-5-trifluoromethyl-pyridin-2-yl)-piperazin-1-yl]-[5-methanesulfonyl-2-(2,2,2-trifluoro-ethylsulfanyl)-phenyl]-methanone and
[4-(2-fluoro-4-methanesulfonyl-phenyl)-piperazin-1-yl]-[5-methanesulfonyl-2-(2,2,2-trifluoro-ethylsulfanyl)-phenyl]-methanone.

Also preferred are compounds of formula I in which R² is —S(O)₂-lower alkyl. Of these, compounds in which R¹ is lower alkyl are preferred. Also preferred, are compound of formula I in which R² is —S(O)₂-lower alkyl and R¹ is lower alkyl substituted by halogen.

In one embodiment, compounds of formula IA are preferred. In particular, preferred compounds are those of formula IA in which R² is —S(O)₂-lower alkyl. Of these compounds, those in which R¹ is lower alkyl are preferred. Also preferred are such compounds where R¹ is lower alkyl substituted by halogen. Alternatively, preferred compounds are those of formula IA in which R² is —S(O)₂NH-lower alkyl.

In another embodiment, compounds of formula IB are preferred. In particular, preferred compounds are those of formula IB in which R² is —S(O)₂-lower alkyl. Of these compounds, those in which R¹ is lower alkyl are preferred. Also preferred are such compounds where R¹ is lower alkyl substituted by halogen. Alternatively, preferred compounds are those of formula IB in which R² is —S(O)₂NH-lower alkyl.

In one embodiment, compounds of formula IC are preferred. In particular, preferred compounds are those of formula IC in which R² is —S(O)₂-lower alkyl. Of these compounds, those in which R¹ is lower alkyl are preferred. Also preferred are such compounds where R¹ is lower alkyl substituted by halogen. Alternatively, preferred compounds are those of formula IC in which R² is —S(O)₂NH-lower alkyl.

The present compounds of formula I and their pharmaceutically acceptable salts can be prepared by methods known in the art, for example, by processes described below, which process comprises
a) reacting a compound of formula 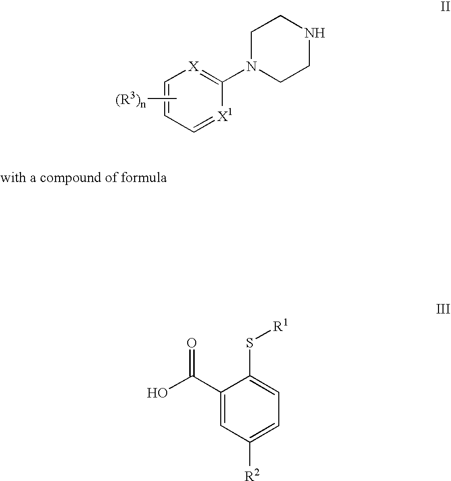
in the presence of an activating agent, such as TBTU (2-(1H-benzotriazole-1-yl)-1,1,3,3-tetramethyluroniumtetrafluoroborate),
to produce a compound of formula 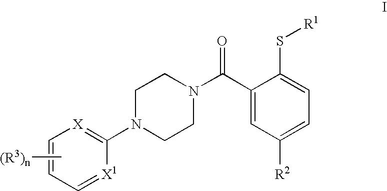
wherein the substituents R¹, R², R³, n, X and X¹ are as defined above, or
b) reacting a compound of formula 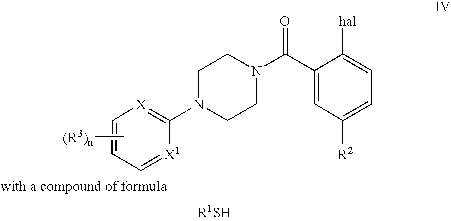
optionally in the presence of a catalyst, such as Cu(I)I or Cu(I)Br, and a base, like potassium carbonate, cesium carbonate or sodium,
to produce a compound of formula 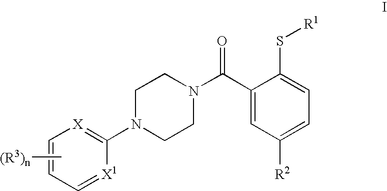
wherein hal is halogen, and the other substituents R¹, R², R³, n, X and X¹ are as defined above, and if desired, converting the compounds obtained into pharmaceutically acceptable acid addition salts.

The compounds of formula I can be prepared in accordance with process variant a) or b) and with the following schemes 1 and 2. The starting materials are either commercially available, are otherwise known in the chemical literature, or can be prepared in accordance with methods well known in the art.

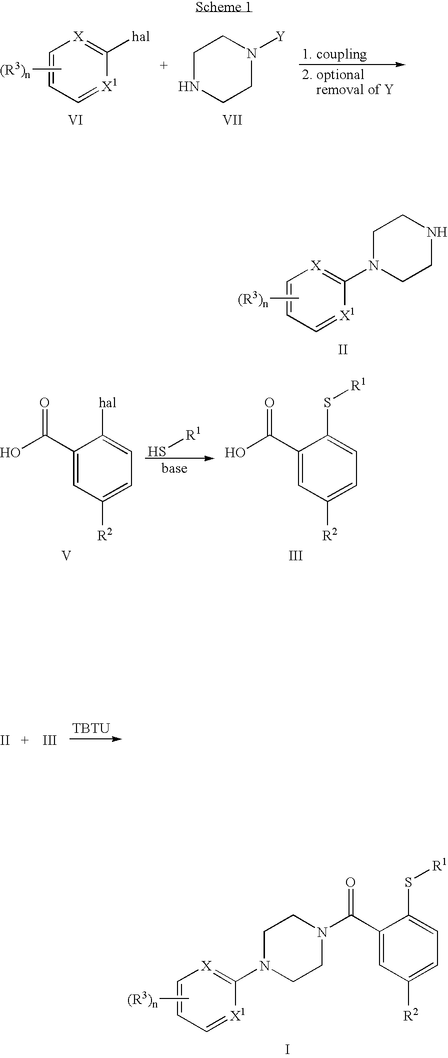

Compounds of general formula I can be prepared by reacting piperazine derivatives of formula II with a corresponding acid of formula III in the presence of an activating agent like TBTU (2-(1H-benzotriazole-1-yl)-1,1,3,3-tetramethyluroniumtetrafluoroborate). The acid of formula III can be prepared by reaction of an acid of formula V with a thiol of formula R¹SH, optionally in the presence of a catalyst, such as Cu(I)I or Cu(I)Br and a base like potassium carbonate, cesium carbonate or sodium. Piperazine derivatives of formula II can be prepared by heating of piperazine of formula VII with the analogous halogen compound of formula VI, optionally in the presence of an organopalladium catalyst. Alternatively, piperazine derivatives of formula II can also be prepared by heating of N-protected piperazine with the analogous halogen compound of formula VI, optionally in the presence of an organopalladium catalyst, followed by cleavage of the protective group. The protective group is typically tert-butoxycarbonyl (Boc).

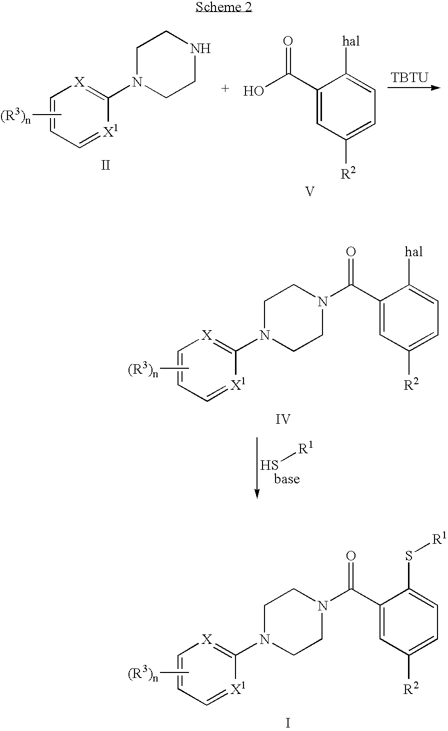

Alternatively, compounds of general formula I can be prepared by reaction of an acyl piperazine of formula IV and a thiol of formula R¹SH, optionally in the presence of a catalyst, such as Cu(I)I or Cu(I)Br and a base like potassium carbonate, cesium carbonate or sodium. Acylpiperazine derivatives of formula IV can be prepared by reaction of an acid of formula V with piperazine derivatives of formula II in the presence of an activating agent like TBTU (2-(1H-benzotriazole-1-yl)-1,1,3,3-tetramethyluroniumtetrafluoroborate).

Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used Racemic mixtures of chiral compounds of formula I can be separated using preparative chiral HPLC.

Salts of Compounds of Formula I

The compounds of formula I can be basic, for example in cases where the compounds contain a basic group such as an aliphatic or aromatic amine moiety. In such cases the compounds of formula I can be converted to a corresponding acid addition salt.

The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or can be brought out of solution with a less polar solvent.

The acid addition salts of the basic compounds of formula I can be converted to the corresponding free bases by treatment with at least a stoichiometric equivalent of a suitable base such as sodium or potassium hydroxide, potassium carbonate, sodium bicarbonate, ammonia, and the like.

The compounds of formula I and their pharmaceutically usable addition salts possess valuable pharmacological properties. Specifically, it has been found that the compounds of the present invention are good inhibitors of the glycine transporter I (GlyT-1).

The compounds were investigated in accordance with the test given hereinafter.

### Solutions and Materials

DMEM complete medium: Nutrient mixture F-12 (Gibco Life-technologies), fetal bovine serum (FBS) 5%, (Gibco life technologies), Penicillin/Streptomycin 1% (Gibco life technologies), Hygromycin 0.6 mg/ml (Gibco life technologies), Glutamine 1 mM Gibco life technologies)

Uptake buffer (UB): 150 mM NaCl, 10 mM Hepes-Tris, pH 7.4, 1 mM CaCl₂, 2.5 mM KCl, 2.5 mM MgSO₄, 10 mM (+) D-glucose.

Flp-in™-CHO (Invitrogen Cat no R758-07) cells stably transfected with mGlyT1b cDNA.

Glycine Uptake Inhibition Assay (mGlyT-1b)

On day 1 mammalian cells, (Flp-in™-CHO), transfected with mGlyT-1b cDNA, were plated at the density of 40,000 cells/well in complete F-12 medium, without hygromycin in 96-well culture plates. On day 2, the medium was aspirated and the cells were washed twice with uptake buffer (UB). The cells were then incubated for 20 min at 22° C. with either (i) no potential competitor, (ii) 10 mM non-radioactive glycine, (iii) a concentration of a potential inhibitor. A range of concentrations of the potential inhibitor was used to generate data for calculating the concentration of inhibitor resulting in 50% of the effect (e.g. IC₅₀, the concentration of the competitor inhibiting glycine uptake of 50%). A solution was then immediately added containing [³H]-glycine 60 nM (11-16 Ci/mmol) and 25 μM non-radioactive glycine. The plates were incubated with gentle shaking and the reaction was stopped by aspiration of the mixture and washing (three times) with ice-cold UB. The cells were lysed with scintillation liquid, shaken 3 hours and the radioactivity in the cells was counted using a scintillation counter.

The prepared compounds have good IC₅₀ (μM) at GlyT-1.

The preferred compounds have an IC₅₀ (μM) at GlyT-1 in the range of 0.006-0.1. Representative compounds are shown in the table below.

| Example No. | IC₅₀ (μM) |
|---|---|
| 3 | 0.03 |
| 6 | 0.04 |
| 7 | 0.08 |
| 11 | 0.07 |
| 14 | 0.10 |
| 18 | 0.10 |
| 22 | 0.015 |
| 23 | 0.018 |
| 25 | 0.006 |
| 27 | 0.007 |

The present invention also provides pharmaceutical compositions containing compounds of the invention, for example compounds of formula I and their pharmaceutically acceptable acid addition salts, and a pharmaceutically acceptable carrier. Such pharmaceutical compositions can be in the form of tablets, coated tablets, dragées, hard and soft gelatine capsules, solutions, emulsions or suspensions. The pharmaceutical compositions also can be in the form of suppositories or injectable solutions.

The pharmaceutical compounds of the invention, in addition to one or more compounds of the invention, contain a pharmaceutically acceptable carrier. Suitable pharmaceutically acceptable carriers include pharmaceutically inert, inorganic and organic carriers. Lactose, corn starch of derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The invention also provides a method for preparing compositions of the invention which comprises bringing one or more compounds of formula I and/or pharmaceutically acceptable acid addition salts thereof and, if desired, one or more other therapeutically valuable substances into a galenical administration form together with one or more therapeutically inert carriers. Compounds of formula I are good inhibitors of the glycine transporter 1 (GlyT-1), and have good selectivity to glycine transporter 2 (GlyT-2) inhibitors. The invention further provides methods for the treatment of diseases related to activation of NMDA receptors via Glyt-1 inhibition, such as psychoses, dysfunction in memory and learning, schizophrenia, dementia and other diseases in which cognitive processes are impaired, such as attention deficit disorders or Alzheimer's disease. In particular, the present invention provides a method for treating schizophrenia, which comprises administering a therapeutically effective amount of a compound of formula I or a pharmaceutically acceptable salt thereof. The invention also provides a method of treating cognitive impairment, which comprises administering a therapeutically effective amount of a compound of formula I or a pharmaceutically acceptable salt thereof. The invention further provides a method for the treatment of Alzheimer's disease, which comprises administering a therapeutically effective amount of a compound of formula I or a pharmaceutically acceptable salt thereof.

The dosage at which compounds of the invention can be administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

Manufacturing Procedure
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Manufacturing Procedure
1. Mix items 1, 2 and 3 in a suitable mixer for 30 minutes.
2. Add items 4 and 5 and mix for 3 minutes.
3. Fill into a suitable capsule.

The following examples illustrate the invention but are not intended to limit its scope.

The following abbreviations were used in the examples:

n-Boc-piperazine: tert-Butyl 1-piperazinecarboxylate,

Oxone®: (potassium peroxymonosulfate) 2 KHSO₅.KHSO₄.K₂SO₄,

TBTU: 2-(1H-benzotriazole-1-yl)-1,1,3,3-tetramethyluroniumtetrafluoroborate;

Synthesis of Intermediates of Formula II

### EXAMPLE A1

### 1-(3-Fluoro-5-trifluoromethyl-pyridin-2-yl)-piperazine

### (a) 4-(3-Fluoro-5-trifluoromethyl-pyridin-2-yl)-piperazine-1-carboxylic acid tert-butyl ester

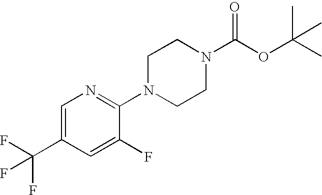

To a mixture of 120 mmol N-Boc-piperazine and 229 mmol potassium carbonate in 300 ml acetonitrile was slowly added a solution of 115 mmol 2,3-difluoro-5-trifluoromethyl-pyridine (EP0104715) in 15 ml acetonitrile. The reaction mixture was refluxed for 2 hours. After such time, the mixture was filtered and the filtrate was concentrated in vacuo. The resulting white solid was dissolved in ethyl acetate, washed with water, dried over sodium sulfate and concentrated to yield the title compound as white solid (94% yield). MS (m/e): 294.2 (M−C₄H₈, 100%)

### (b) 1-(3-Fluoro-5-trifluoromethyl-pyridin-2-yl)-piperazine

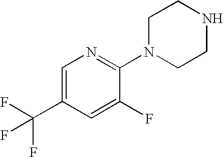

To a solution of 107.3 mmol 4-(3-fluoro-5-trifluoromethyl-pyridin-2-yl)-piperazine-1-carboxylic acid tert-butyl ester in 376 ml dichloromethane was added 41 ml trifluoroacetic acid and the reaction mixture was refluxed for 16 hours. After such time the reaction mixture was concentrated in vacuo and the residue was dissolved in water. The aqueous phase was basified with 5N NaOH and extracted with dichloromethane. The combined extracts were washed with brine, dried over sodium sulfate and concentrated in vacuo to yield the title compound as a light yellow solid (99% yield). MS (m/e): 250.2 (MH⁺, 100%)

### EXAMPLE A2

### 2-piperazin-1-yl-5-trifluoromethyl-pyrimidine

### a) 2-(4-Benzyl-piperazin-1-yl)-5-trifluoromethyl-pyrimidine

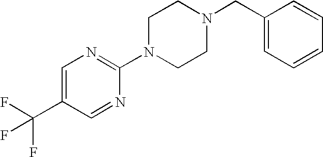

To a solution of 2.60 mmol (3-dimethylamino-2-trifluoromethyl-allylidene)-dimethyl-ammonium chloride (prepared as in Tetrahedron. Lett. 1996, 37, 1829) in 10 ml acetonitrile was added 3.03 mmol 4-benzyl-piperazine-1-carboxamidine hydrochloride (prepared as in Tetrahedron. Lett. 2002, 43, 7105) and 6.25 mmol triethylamine and the reaction mixture was stirred for 3 hours at room temperature. After such time the reaction mixture was concentrated in vacuo and purified by column chromatography to yield the title compound as a light yellow solid (0.79 g). MS (m/e): 323.4 (M+H⁺).

### b) 2-piperazin-1-yl-5-trifluoromethyl-pyrimidine

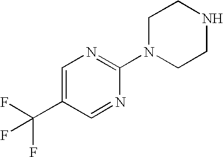

To a solution of 2-(4-benzyl-piperazin-1-yl)-5-trifluoromethyl-pyrimidine (0.63 g) in methanol was added palladium on charcoal (5% w/w) and the reaction mixture was heated at 60° C. under a hydrogen atmosphere. The reaction mixture was then allowed to cool to room temperature, the catalyst was filtered off, and the solvent was removed in vacuo to yield the title compound as a white solid (0.41 g). MS (m/e): 233.1 (M+H⁺).

### EXAMPLE A3

### 1-(3-Fluoro-4-piperazin-1-yl-phenyl)-ethanone

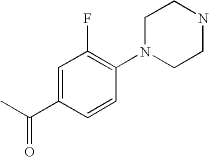

This compound (CAS: 189763-57-3) was prepared according to WO 9714690. MS (m/e): 223.2 (M+H⁺).

### EXAMPLE A4

### 1-(2-Chloro-4-nitro-phenyl)-piperazine

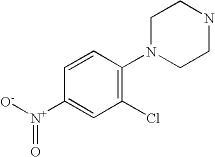

To a mixture of 43.4 mmol of piperazine in 11 ml DMF was slowly added a solution of 8.6 mmol of 1,2-dichloro-4-nitrobenzene in 15 ml DMF. The reaction mixture was heated at 100° C. for 2.5 hours. After such time, the mixture was filtered and the filtrate was concentrated in vacuo. The residue was diluted with dichloromethane (25 mL) and washed with saturated aqueous solution of NaHCO₃. The organic phase was then dried over sodium sulfate and concentrated in vacuo. The residue was then purified by column chromatography (SiO₂, CH₂Cl₂-MeOH 0-10%) to yield the title compound as yellow solid (766 mg, 37% yield). MS (m/e): 242.3 (M+H⁺, 100%).

### EXAMPLE A5

### 1-(5-Methyl-pyridin-2-yl)-piperazine

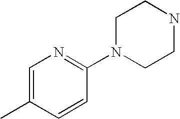

To a solution of 23 mmol 6-chloro-3-methylpyridine in 12 ml DMA and 30 ml water was added 115 mmol piperazine. The reaction mixture was refluxed for 4 days. After such time, 2 N aq sodium carbonate was added, the reaction mixture was extracted with dichloromethane and concentrated in vacuo. The residue was purified on silica gel (dichloromethane/MeOH 1/1) to yield the title compound as white solid (17% yield). MS (m/e): 178 (M+H⁺, 100%)

### Synthesis of Compounds of Formula I

### EXAMPLE 1

### 4-[4-(5-Methanesulfonyl-2-methylsulfanyl-benzoyl)-piperazin-1-yl]-benzonitrile

### a) 2-Iodo-5-methanesulfonyl-benzoic acid

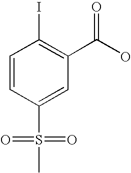

To a suspension of 3.0 mmol 2-amino-5-methanesulfonyl-benzoic acid (WO2003091245) in a mixture of 1.7 ml sulfuric acid and 1.7 ml water was added dropwise a solution of 3.92 mmol sodium nitrite in 1.7 ml water at such a rate that the temperature did not exceed 3° C. The mixture was stirred at 0° C. for 1 hour. A solution of 3.0 mmol potassium iodide in 1.7 ml water was added dropwise at 0° C. The brown suspension was allowed to warm to room temperature and stirred for 30 minutes. Excess iodine was destroyed by addition of a few drops of a sodium hydrogensulfite solution. The solid was filtered, washed with water and dried (HV, 50° C., 1 hour) to yield the title compound. MS (m/e): 325.0 ([M−H]⁻, 100%)

### b) 4-[4-(2-Iodo-5-methanesulfonyl-benzoyl)-piperazin-1-yl]-benzonitrile

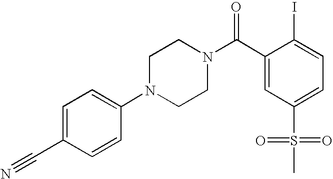

To a solution of 6.1 mmol 2-iodo-5-methanesulfonyl-benzoic acid in 20 ml dimethyl formamide were added successively 6.75 mmol TBTU, 43 mmol N-ethyldiisopropylamine and 6.75 mmol 4-piperazin-1-yl-benzonitrile (commercially available, e.g. from Fluka). The reaction mixture was stirred at 20° C. for 1.5 h and then concentrated in vacuo. Addition of 200 ml water followed by filtration yielded the crude product which was recrystallized from methanol to afford the title compound as a white solid (yield 87%). MS (m/e): 495.9 (M+H⁺, 100%).

### c) 4-[4-(5-Methanesulfonyl-2-methylsulfanyl-benzoyl)-piperazin-1-yl]-benzonitrile

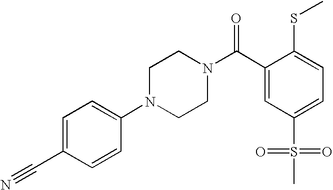

A solution of 0.2 mmol 4-[4-(2-iodo-5-methanesulfonyl-benzoyl)-piperazin-1-yl]-benzonitrile in 2 ml dimethylformamide was treated with 0.2 mmol of sodium methanethiolate and held at 50° C. for 2 h, at 80° C. for 2 h and at 140° C. for 1 h. The reaction mixture was then concentrated and the product purified by chromatography (dichloromethane/methanol) to afford the title compound as a colorless foam. MS (m/e): 474.2 ([M+CH₃COO⁻]⁻, 100%).

### EXAMPLE 2

### (2-Isopropylsulfanyl-5-methanesulfonyl-phenyl)-[4-(4-methanesulfonyl-phenyl)-piperazin-1-yl]-methanone

### a) (2-Iodo-5-methanesulfonyl-phenyl)-[4-(4-methanesulfonyl-phenyl)-piperazin-1-yl]-methanone

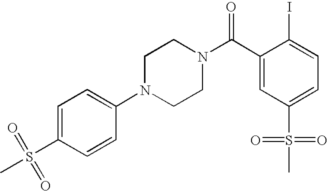

Prepared in analogy to example 1b) from 1-(4-methanesulfonyl-phenyl)-piperazine (commercially available, e.g. from Peakdale) and 2-iodo-5-methanesulfonyl-benzoic acid (Example 1a). White solid. MS (m/e): 566.2 (M+NH₄⁺, 100%).

### b) (2-Isopropylsulfanyl-5-methanesulfonyl-phenyl)-[4-(4-methanesulfonyl-phenyl)-piperazin-1-yl]-methanone

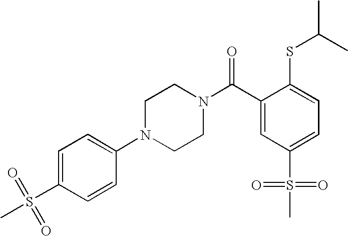

A solution of 0.9 mmol of (2-iodo-5-methanesulfonyl-phenyl)-[4-(4-methanesulfonyl-phenyl)-piperazin-1-yl]-methanone in 10 ml dimethylformamide was treated with 1.2 mmol of 2-propanethiol, 1.6 mmol of potassium carbonate, 0.1 mmol of copper powder and 1 mg of copper(I)-iodide and refluxed for 4 h. Concentration followed by dilution with 100 ml water yielded a dark solid which was purified by chromatography (ethyl acetate) to give the title compound as a colorless foam. MS (m/e): 555.2 ([M+CH₃COO⁻]⁻, 100%).

### EXAMPLE 3

### [4-(3-Fluoro-5-trifluoromethyl-pyridin-2-yl)-piperazin-1-yl]-(2-isopropylsulfanyl-5-methanesulfonyl-phenyl)-methanone

### a) 2-Fluoro-5-methylsulfanyl-benzoic acid

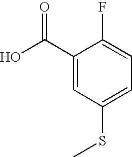

The title compound was prepared by following the procedure described in: Journal of Organometallic Chemistry 1991, 419(1-2), 1-8.

### b) 2-Fluoro-5-methanesulfonyl-benzoic acid

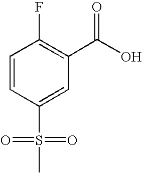

To 2.68 mmol 2-fluoro-5-methanesulfanyl-benzoic acid in 5 ml methanol at 0° C. was added 8.05 mmol Oxone® and the mixture was allowed to stir at RT for 72 h. The precipitate was filtered off and the filtrate was concentrated under reduced pressure. The residue was treated with water and extracted 3 times with 400 ml dichloromethane. The combined organic phases were dried over sodium sulfate. Evaporation under reduced pressure yielded the title compound as a white crystalline solid (yield 79%). MS (m/e): 217.2 (M−H⁺, 100%).

### c) 2-Isopropylsulfanyl-5-methanesulfonyl-benzoic acid

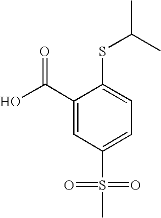

To a solution of 4.58 mmol 2-fluoro-5-methanesulfonyl-benzoic acid in 6 ml N,N-dimethylacetamide were added 15.2 mol cesium carbonate and 10.1 mmol 2-propanethiol and the mixture was stirred at 90° C. for 3 h. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as a light yellow liquid which was used in the next step without further purification (yield 99%). EI-MS (m/e): 274.1 (M⁺, 35%), 232.1 ([M−C₃H₆]⁺, 30%, 214.1 (M−C₃H₆—H₂O)⁺, 100%).

### d) [4-(3-Fluoro-5-trifluoromethyl-pyridin-2-yl)-piperazin-1-yl]-(2-isopropylsulfanyl-5-methanesulfonyl-phenyl)-methanone

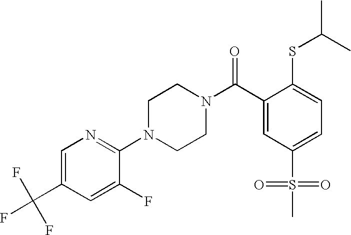

To a solution of 0.51 mmol 2-isopropylsulfanyl-5-methanesulfonyl-benzoic acid in 5 ml tetrahydrofuran were added successively 0.38 mmol TBTU, 1.02 mmol N-ethyldiisopropylamine and 0.30 mmol 1-(3-fluoro-5-trifluoromethyl-pyridin-2-yl)-piperazine (Example A1). The reaction mixture was stirred at 45° C. for 16 h and then concentrated in vacuo. Chromatography (SiO₂, ethyl acetate/heptane) afforded the title compound as a light yellow gum (yield 56%). MS (m/e): 506.1 (M+H⁺, 100%).

### EXAMPLE 4

### (2-Isopropylsulfanyl-5-methanesulfonyl-phenyl)-[4-(5-trifluoromethyl-pyrimidin-2-yl)-piperazin-1-yl]-methanone

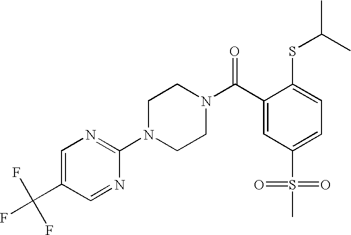

Prepared in analogy to example 3 (d) from 2-isopropylsulfanyl-5-methanesulfonyl-benzoic acid and 2-piperazin-1-yl-5-trifluoromethyl-pyrimidine (Example A2). The crude material was purified by chromatography (SiO₂, ethyl acetate/heptane) to yield the title compound as a brown solid (yield 55%). MS (m/e): 489.0 (M+H⁺, 100%).

### EXAMPLE 5

### (2-Isopropylsulfanyl-5-methanesulfonyl-phenyl)-[4-(4-trifluoromethyl-phenyl)-piperazin-1-yl]-methanone

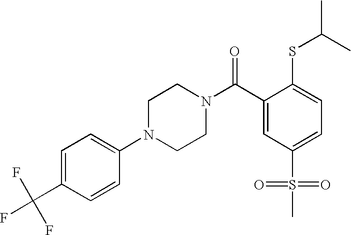

Prepared in analogy to example 3 (d) from 2-isopropylsulfanyl-5-methanesulfonyl-benzoic acid and 1-(4-trifluoromethyl-phenyl)-piperazine (commercially available, e.g. from Fluorochem). The crude material was purified by chromatography (SiO₂, ethyl acetate/heptane) followed by trituration in pentane to yield the title compound as a light yellow foam (yield 40%). MS (m/e): 487.3 (M+H⁺, 100%).

### EXAMPLE 6

### [4-(2-Fluoro-4-methanesulfonyl-phenyl)-piperazin-1-yl]-(2-isopropylsulfanyl-5-methanesulfonyl-phenyl)-methanone

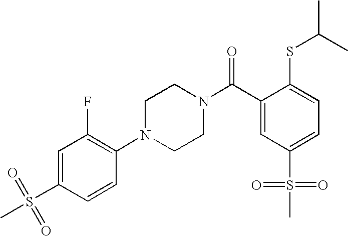

Prepared in analogy to example 3 (d) from 2-isopropylsulfanyl-5-methanesulfonyl-benzoic acid and 1-(2-fluoro-4-methanesulfonyl-phenyl)-piperazine (commercially available, e.g. from Peakdale). The crude material was purified by chromatography (SiO₂, ethyl acetate/heptane) to yield the title compound as a yellow solid (yield 28%). MS (m/e): 515.0 (M+H⁺, 100%).

### EXAMPLE 7

### (2-Ethylsulfanyl-5-methanesulfonyl-phenyl)-[4-(3-fluoro-5-trifluoromethyl-pyridin-2-yl)-piperazin-1-yl]-methanone

### a) 2-Ethylsulfanyl-5-methanesulfonyl-benzoic acid

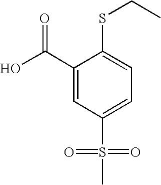

To a solution of 4.58 mmol 2-fluoro-5-methanesulfonyl-benzoic acid in 6 ml N,N-dimethylformamide were added 13.8 mol cesium carbonate and 9.25 mmol ethanethiol and the mixture was stirred at 90° C. for 30 min. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as a white solid which was used in the next step without further purification (yield 99%). MS (m/e): 259.0 ([M−H]⁻, 100%).

### b) (2-Ethylsulfanyl-5-methanesulfonyl-phenyl)-[4-(3-fluoro-5-trifluoromethyl-pyridin-2-yl)-piperazin-1-yl]-methanone

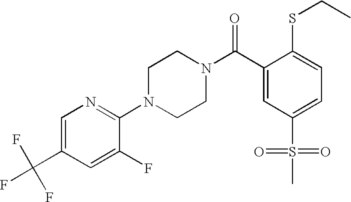

To a solution of 0.27 mmol 2-ethylsulfanyl-5-methanesulfonyl-benzoic acid in 5 ml tetrahydrofuran were added successively 0.40 mmol TBTU, 1.08 mmol N-ethyldiisopropylamine and 0.32 mmol 1-(3-fluoro-5-trifluoromethyl-pyridin-2-yl)-piperazine (Example A1). The reaction mixture was stirred at 45° C. for 16 h and then concentrated in vacuo. Chromatography (SiO₂, ethyl acetate/heptane) afforded the title compound as a light yellow solid (yield 61%). MS (m/e): 492.0 (M+H⁺, 100%).

### EXAMPLE 8

### (2-Ethylsulfanyl-5-methanesulfonyl-phenyl)-[4-(5-trifluoromethyl-pyrimidin-2-yl)-piperazin-1-yl]-methanone

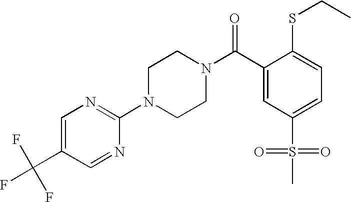

Prepared in analogy to example 7 (b) from 2-ethylsulfanyl-5-methanesulfonyl-benzoic acid and 2-piperazin-1-yl-5-trifluoromethyl-pyrimidine (Example A2). The crude material was purified by chromatography (SiO₂, ethyl acetate/heptane) to yield the title compound as a light yellow solid (yield 52%). MS (m/e): 475.0 (M+H⁺, 100%).

### EXAMPLE 9

### (2-Ethylsulfanyl-5-methanesulfonyl-phenyl)-[4-(4-trifluoromethyl-phenyl)-piperazin-1-yl]-methanone

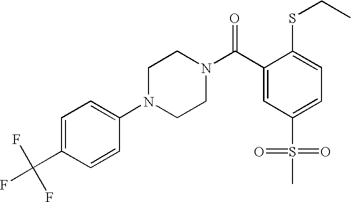

Prepared in analogy to example 7 (b) from 2-ethylsulfanyl-5-methanesulfonyl-benzoic acid and 1-(4-trifluoromethyl-phenyl)-piperazine (commercially available, e.g. from Fluorochem). The crude material was purified by chromatography (SiO₂, ethyl acetate/heptane) to yield the title compound as a white solid (yield 60%). MS (m/e): 473.0 (M+H⁺, 100%).

### EXAMPLE 10

### (2-Ethylsulfanyl-5-methanesulfonyl-phenyl)-[4-(2-fluoro-4-methanesulfonyl-phenyl)-piperazin-1-yl]-methanone

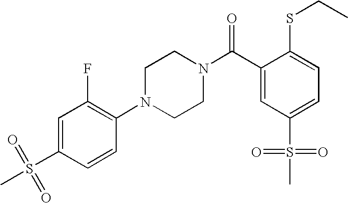

Prepared in analogy to example 7 (b) from 2-ethylsulfanyl-5-methanesulfonyl-benzoic acid and 1-(2-fluoro-4-methanesulfonyl-phenyl)-piperazine (commercially available, e.g. from Peakdale). The crude material was purified by chromatography (SiO₂, ethyl acetate/heptane) to yield the title compound as a light brown solid (yield 74%). MS (m/e): 501.0 (M+H⁺, 100%).

### EXAMPLE 11

### [4-(3-Fluoro-5-trifluoromethyl-pyridin-2-yl)-piperazin-1-yl]-[5-methanesulfonyl-2-(2,2,2-trifluoro-ethylsulfanyl)-phenyl]-methanone

### a) 5-Methanesulfonyl-2-(2,2,2-trifluoro-ethylsulfanyl)-benzoic acid

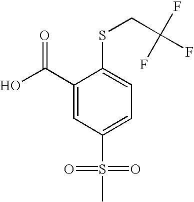

To a solution of 4.58 mmol 2-fluoro-5-methanesulfonyl-benzoic acid in 6 ml N,N-dimethylformamide were added 13.8 mol cesium carbonate and 9.16 mmol 2,2,2-trifluoro-ethanethiol and the mixture was stirred at 90° C. for 30 min. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as a red-brown solid which was used in the next step without further purification (yield 99%). MS (m/e): 312.9 ([M−H]⁻, 100%).

### b) [4-(3-Fluoro-5-trifluoromethyl-pyridin-2-yl)-piperazin-1-yl]-[5-methanesulfonyl-2-(2,2,2-trifluoro-ethylsulfanyl)-phenyl]-methanone

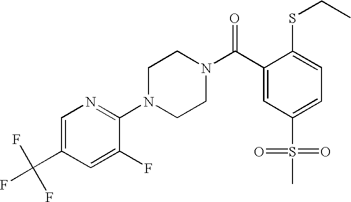

To a solution of 0.22 mmol 5-methanesulfonyl-2-(2,2,2-trifluoro-ethylsulfanyl)-benzoic acid in 5 ml tetrahydrofuran were added successively 0.33 mmol TBTU, 0.89 mmol N-ethyldiisopropylamine and 0.27 mmol 1-(3-fluoro-5-trifluoromethyl-pyridin-2-yl)-piperazine (Example A1). The reaction mixture was stirred at 40° C. for 16 h and then concentrated in vacuo. Chromatography (SiO₂, ethyl acetate/heptane) afforded the title compound as a white foam (yield 47%). MS (m/e): 546.0 (M+H⁺, 100%).

### EXAMPLE 12

### [5-Methanesulfonyl-2-(2,2,2-trifluoro-ethylsulfanyl)-phenyl]-[4-(5-trifluoromethyl-pyrimidin-2-yl)-piperazin-1-yl]-methanone

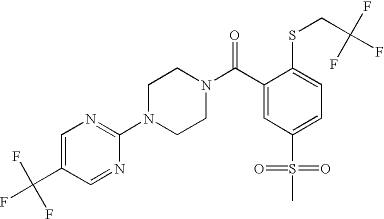

Prepared in analogy to example 11 (b) from 5-methanesulfonyl-2-(2,2,2-trifluoro-ethylsulfanyl)-benzoic acid and 2-piperazin-1-yl-5-trifluoromethyl-pyrimidine (Example A2). The crude material was purified by chromatography (SiO₂, ethyl acetate/heptane) to yield the title compound as a white foam (yield 58%). MS (m/e): 529.0 (M+H⁺, 100%).

### EXAMPLE 13

### [5-Methanesulfonyl-2-(2,2,2-trifluoro-ethylsulfanyl)-phenyl]-[4-(4-trifluoromethyl-phenyl)-piperazin-1-yl]-methanone

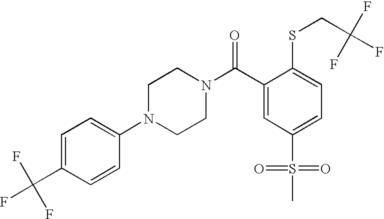

Prepared in analogy to example 11 (b) from 5-methanesulfonyl-2-(2,2,2-trifluoro-ethylsulfanyl)-benzoic acid and 1-(4-trifluoromethyl-phenyl)-piperazine (commercially available, e.g. from Fluorochem). The crude material was purified by chromatography (SiO₂, ethyl acetate/heptane) to yield the title compound as a brown solid (yield 76%). MS (m/e): 527.2 (M+H⁺, 100%).

### EXAMPLE 14

### [4-(2-Fluoro-4-methanesulfonyl-phenyl)-piperazin-1-yl]-[5-methanesulfonyl-2-(2,2,2-trifluoro-ethylsulfanyl)-phenyl]-methanone

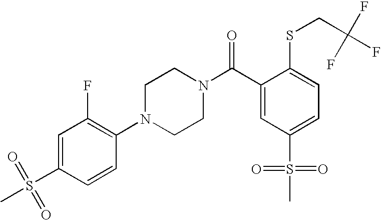

Prepared in analogy to example 11 (b) from 5-methanesulfonyl-2-(2,2,2-trifluoro-ethylsulfanyl)-benzoic acid and 1-(2-fluoro-4-methanesulfonyl-phenyl)-piperazine (commercially available, e.g. from Peakdale). The crude material was purified by chromatography (SiO₂, ethyl acetate/heptane) to yield the title compound as a light brown solid (yield 74%). MS (m/e): 555.0 (M+H⁺, 100%).

### EXAMPLE 15

### [4-(3-Fluoro-5-trifluoromethyl-pyridin-2-yl)-piperazin-1-yl]-(2-isobutylsulfanyl-5-methanesulfonyl-phenyl)-methanone

### a) 2-Isobutylsulfanyl-5-methanesulfonyl-benzoic acid

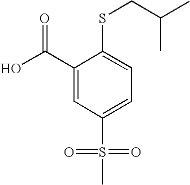

To a solution of 4.58 mmol 2-fluoro-5-methanesulfonyl-benzoic acid in 6 ml N,N-dimethylformamide were added 13.8 mol cesium carbonate and 9.97 mmol 2-methyl-1-propanethiol and the mixture was stirred at 90° C. for 30 min. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as a white solid which was used in the next step without further purification (yield 99%). MS (m/e): 287.0 ([M−H]⁻, 100%).

### b) [4-(3-Fluoro-5-trifluoromethyl-pyridin-2-yl)-piperazin-1-yl]-(2-isobutylsulfanyl-5-methanesulfonyl-phenyl)-methanone

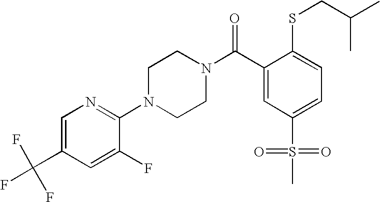

To a solution of 0.24 mmol 2-isobutylsulfanyl-5-methanesulfonyl-benzoic acid in 5 ml tetrahydrofuran were added successively 0.36 mmol TBTU, 0.97 mmol N-ethyldiisopropylamine and 0.29 mmol 1-(3-fluoro-5-trifluoromethyl-pyridin-2-yl)-piperazine (Example A1). The reaction mixture was stirred at 45° C. for 16 h and then concentrated in vacuo. Chromatography (SiO₂, ethyl acetate/heptane) afforded the title compound as a light brown solid (yield 57%). MS (m/e): 520.0 (M+H⁺, 100%).

### EXAMPLE 16

### (2-Isobutylsulfanyl-5-methanesulfonyl-phenyl)-[4-(5-trifluoromethyl-pyrimidin-2-yl)-piperazin-1-yl]-methanone

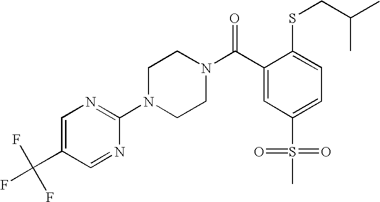

Prepared in analogy to example 15 (b) from 2-isobutylsulfanyl-5-methanesulfonyl-benzoic acid and 2-piperazin-1-yl-5-trifluoromethyl-pyrimidine (Example A2). The crude material was purified by chromatography (SiO₂, ethyl acetate/heptane) to yield the title compound as a white foam (yield 77%). MS (m/e): 503.0 (M+H⁺, 100%).

### EXAMPLE 17

### (2-Isobutylsulfanyl-5-methanesulfonyl-phenyl)-[4-(4-trifluoromethyl-phenyl)-piperazin-1-yl]-methanone

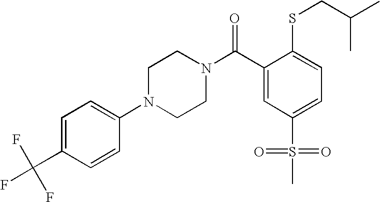

Prepared in analogy to example 15 (b) from 2-isobutylsulfanyl-5-methanesulfonyl-benzoic acid and 1-(4-trifluoromethyl-phenyl)-piperazine (commercially available, e.g. from Fluorochem). The crude material was purified by chromatography (SiO₂, ethyl acetate/heptane) to yield the title compound as a brown solid (yield 99%). MS (m/e): 501.0 (M+H⁺, 100%).

### EXAMPLE 18

### [4-(2-Fluoro-4-methanesulfonyl-phenyl)-piperazin-1-yl]-(2-isobutylsulfanyl-5-methanesulfonyl-phenyl)-methanone

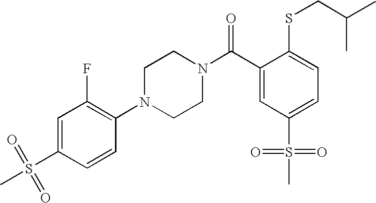

Prepared in analogy to example 15 (b) from 2-isobutylsulfanyl-5-methanesulfonyl-benzoic acid and 1-(2-fluoro-4-methanesulfonyl-phenyl)-piperazine (commercially available, e.g. from Peakdale). The crude material was purified by chromatography (SiO₂, ethyl acetate/heptane) to yield the title compound as an orange foam (yield 99%). MS (m/e): 529.0 (M+H⁺, 100%).

### EXAMPLE 19

### [4-(2-Fluoro-4-methanesulfonyl-phenyl)-piperazin-1-yl]-(5-methanesulfonyl-2-methylsulfanyl-phenyl)-methanone

### a) 5-Methanesulfonyl-2-methylsulfanyl-benzoic acid

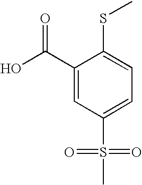

To a solution of 4.58 mmol 2-fluoro-5-methanesulfonyl-benzoic acid in 6 ml N,N-dimethylformamide were added 13.8 mol cesium carbonate and 10.0 mmol sodium methanethiolate and the mixture was stirred at 90° C. for 30 min. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as a colourless oil which was used in the next step without further purification (yield 99%). MS (m/e): 244.9 ([M−H]⁻, 100%).

### b) [4-(2-Fluoro-4-methanesulfonyl-phenyl)-piperazin-1-yl]-(5-methanesulfonyl-2-methylsulfanyl-phenyl)-methanone

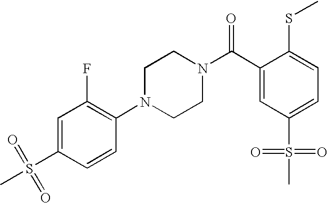

To a solution of 0.28 mmol 5-methanesulfonyl-2-methylsulfanyl-benzoic acid in 5 ml tetrahydrofuran were added successively 0.43 mmol TBTU, 1.14 mmol N-ethyldiisopropylamine and 0.34 mmol 1-(2-fluoro-4-methanesulfonyl-phenyl)-piperazine (commercially available, e.g. from Peakdale). The reaction mixture was stirred at 45° C. for 16 h and then concentrated in vacuo. Chromatography (SiO₂, ethyl acetate/heptane) afforded the title compound as a light brown solid (yield 77%). MS (m/e): 487.3 (M+H⁺, 100%).

### EXAMPLE 20

### [4-(3-Fluoro-5-trifluoromethyl-pyridin-2-yl)-piperazin-1-yl]-(5-methanesulfonyl-2-methylsulfanyl-phenyl)-methanone

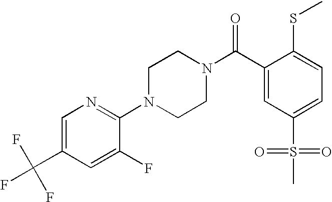

Prepared in analogy to example 19 (b) from 5-methanesulfonyl-2-methylsulfanyl-benzoic acid and 1-(3-fluoro-5-trifluoromethyl-pyridin-2-yl)-piperazine (Example A1). The crude material was purified by chromatography (SiO₂, ethyl acetate/heptane) to yield the title compound as an orange foam (yield 99%). MS (m/e): 478.0 (M+H⁺, 100%).

### EXAMPLE 21

### (5-Methanesulfonyl-2-methylsulfanyl-phenyl)-[4-(4-trifluoromethyl-phenyl)-piperazin-1-yl]-methanone

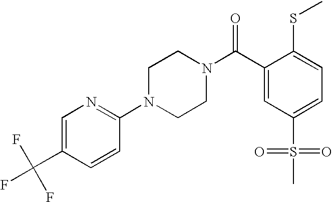

Prepared in analogy to example 19 (b) from 5-methanesulfonyl-2-methylsulfanyl-benzoic acid and 1-(4-trifluoromethyl-phenyl)-piperazine (commercially available, e.g. from Fluorochem). The crude material was purified by chromatography (SiO₂, ethyl acetate/heptane) to yield the title compound as brown foam (yield 99%). MS (m/e): 459.1 (M+H⁺, 100%).

### EXAMPLE 22

### 1-{3-Fluoro-4-[4-(2-isopropylsulfanyl-5-methanesulfonyl-benzoyl)-piperazin-1-yl]-phenyl}-ethanone

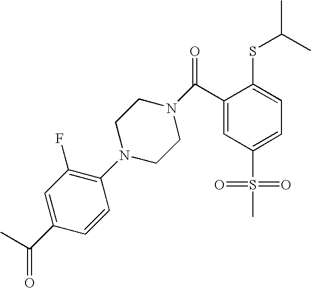

Prepared in analogy to example 3 (d) from 2-isopropylsulfanyl-5-methanesulfonyl-benzoic acid and 1-(3-fluoro-4-piperazin-1-yl-phenyl)-ethanone (Example A3). The crude material was purified by chromatography (SiO₂, methanol/dichloromethane) to yield the title compound as a white solid (yield 13%). MS (m/e): 479.3 (M+H⁺, 100%).

### EXAMPLE 23

### [4-(2-Chloro-4-nitro-phenyl)-piperazin-1-yl]-(2-isopropylsulfanyl-5-methanesulfonyl-phenyl)-methanone

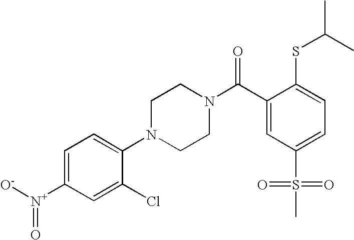

Prepared in analogy to example 3 (d) from 2-isopropylsulfanyl-5-methanesulfonyl-benzoic acid and 1-(2-chloro-4-nitro-phenyl)-piperazine (Example A4). The crude material was purified by chromatography (SiO₂, ethyl acetate/heptane) to yield the title compound as an orange solid (yield 86%). MS (m/e): 500.2 ({³⁷Cl]M+H⁺, 59%), 498.2 ({³⁵Cl]M+H⁺, 100%).

### EXAMPLE 24

### (2-Isopropylsulfanyl-5-methanesulfonyl-phenyl)-[4-(5-methyl-pyridin-2-yl)-piperazin-1-yl]-methanone

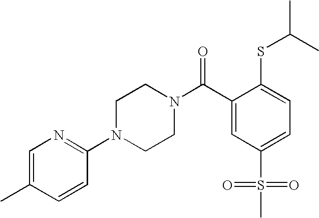

Prepared in analogy to example 3 (d) from 2-isopropylsulfanyl-5-methanesulfonyl-benzoic acid and 1-(5-methyl-pyridin-2-yl)-piperazine (Example A5). The crude material was purified by chromatography (SiO₂, ethyl acetate/heptane) to yield the title compound as an off-white solid (yield 24%). MS (m/e): 434.1 (M+H⁺, 100%).

### EXAMPLE 25

### 4-Isopropylsulfanyl-N-methyl-3-[4-(4-trifluoromethyl-phenyl)-piperazine-1-carbonyl]-benzenesulfonamide

### a) 2-Chloro-5-methylsulfamoyl-benzoic acid

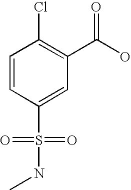

To 41.9 mmol 2-chloro-5-(fluorosulfonyl)sulfanyl-benzoic acid in 100 ml dioxane was added dropwise 420 mmol methylamine solution (41% aqueous) and the mixture was stirred at RT for 2 h. An excess of 2 M aqueous hydrochloric acid was then added to the reaction mixture and the resulting mixture was then concentrated in vacuo. The residue was treated with water and extracted 3 times with ethyl acetate. The combined organic phases were dried over sodium sulfate. Evaporation under reduced pressure yielded the title compound as a white crystalline solid (yield 96%). MS (m/e): 248.1 ([M−H]⁻, 100%).

### b) 2-Isopropylsulfanyl-5-methylsulfamoyl-benzoic acid

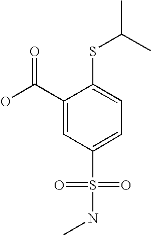

To a solution of 1.99 mmol 2-chloro-5-methylsulfamoyl-benzoic acid in 3 ml N,N-dimethylacetamide were added 6.37 mmol cesium carbonate and 4.39 mmol 2-propanethiol and the mixture was stirred at 100° C. for 3 h. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as an off-white solid which was used in the next step without further purification (yield 85%). MS (m/e): 288.1 ([M−H]⁻, 100%).

### c) 4-Isopropylsulfanyl-N-methyl-3-[4-(4-trifluoromethyl-phenyl)-piperazine-1-carbonyl]-benzenesulfonamide

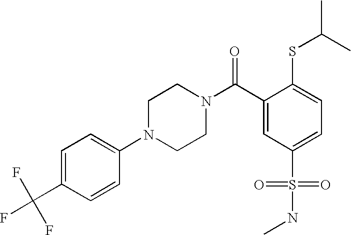

To a solution of 0.17 mmol 2-isopropylsulfanyl-5-methylsulfamoyl-benzoic acid in 3 ml tetrahydrofuran were added successively 0.16 mmol TBTU, 0.42 mmol N-ethyldiisopropylamine and 0.11 mmol 1-(4-trifluoromethylphenyl)-piperazine (commercially available, e.g. from Fluorochem). The reaction mixture was stirred at 35° C. for 16 h and then concentrated in vacuo. Chromatography (SiO₂, ethyl acetate/heptane) followed by trituration in pentane afforded the title compound as a white solid (yield 66%). MS (m/e): 502.1 (M+H⁺, 100%).

### EXAMPLE 26

### 4-Isopropylsulfanyl-3-[4-(4-trifluoromethyl-phenyl)-piperazine-1-carbonyl]-benzonitrile

### a) 5-Cyano-2-isopropylsulfanyl-benzoic acid

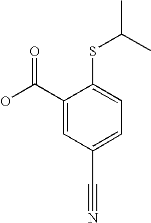

To a solution of 1.45 mmol 5-cyano-2-iodo-benzoic acid (CAS: 219841-92-6, prepared as described in WO2005014563) in 3 ml N,N-dimethylacetamide were added 4.91 mmol cesium carbonate and 3.19 mmol 2-propanethiol and the mixture was stirred at 120° C. for 4 h. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as an off-white solid which was used in the next step without further purification (yield 97%). MS (m/e): 220.1 ([M−H]⁻, 100%).

### b) 4-Isopropylsulfanyl-3-[4-(4-trifluoromethyl-phenyl)-piperazine-1-carbonyl]-benzonitrile

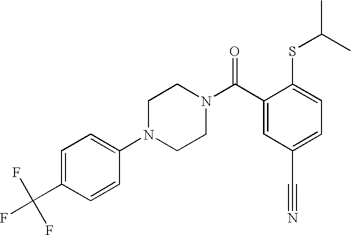

To a solution of 0.32 mmol 5-cyano-2-isopropylsulfanyl-benzoic acid in 5 ml tetrahydrofuran were added successively 0.31 mmol TBTU, 0.84 mmol N-ethyldiisopropylamine and 0.22 mmol 1-(4-trifluoromethylphenyl)-piperazine (commercially available, e.g. from Fluorochem). The reaction mixture was stirred at 35° C. for 16 h and then concentrated in vacuo. Chromatography (SiO₂, ethyl acetate/heptane) followed by trituration in pentane afforded the title compound as an off-white solid (yield 94%). MS (m/e): 434.4 (M+H⁺, 100%).

### EXAMPLE 27

### (2-Isopropylsulfanyl-5-nitro-phenyl)-[4-(4-trifluoromethyl-phenyl)-piperazin-1-yl]-methanone

### a) 2-Isopropylsulfanyl-5-nitro-benzoic acid

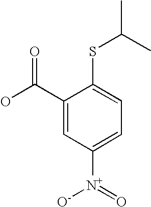

To a solution of 2.67 mmol 5-fluoro-2-nitro-benzoic acid (commercially available, e.g. from Aldrich) in 3 ml N,N-dimethylacetamide were added 8.58 mmol cesium carbonate and 5.83 mmol 2-propanethiol and the mixture was stirred at 35° C. for 4 h. The reaction mixture was then cooled to room temperature and acidified to pH1 by addition of hydrochloric acid before being extracted three times with ethyl acetate. The combined organic phases were dried over sodium sulfate and concentrated in vacuo to afford the title compound as an off-white solid which was used in the next step without further purification (yield 74%). MS (m/e): 240.3 ([M−H]⁻, 100%).

### b) (2-Isopropylsulfanyl-5-nitro-phenyl)-[4-(4-trifluoromethyl-phenyl)-piperazin-1-yl]-methanone

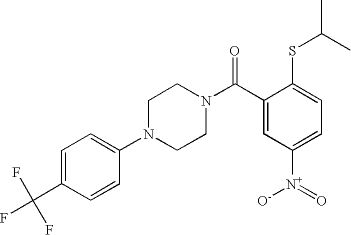

To a solution of 0.31 mmol 2-isopropylsulfanyl-5-nitro-benzoic acid in 5 ml tetrahydrofuran were added successively 0.31 mmol TBTU, 0.84 mmol N-ethyldiisopropylamine and 0.22 mmol 1-(4-trifluoromethylphenyl)-piperazine (commercially available, e.g. from Fluorochem). The reaction mixture was stirred at 35° C. for 16 h and then concentrated in vacuo. Chromatography (SiO₂, ethyl acetate/heptane) followed by trituration in pentane afforded the title compound as a yellow solid (yield 83%). MS (m/e): 454.4 (M+H⁺, 100%).

1. A compound of formula I 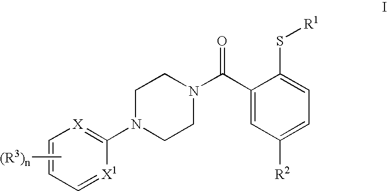
wherein
R¹ is lower alkyl or lower alkyl substituted by halogen;
R² is —S(O)₂-lower alkyl, —S(O)₂NH-lower alkyl, NO₂ or CN;
R³ is halogen, CN, lower alkyl, lower alkyl substituted by halogen, NO₂, —C(O)-lower alkyl or S(O)₂-lower alkyl;
X and X¹ are each independently CR⁴ or N;
R⁴ is hydrogen or halogen; and
n is 0, 1 or 2;
or a pharmaceutically acceptable acid addition salt thereof.

2. A compound of claim 1 having formula I-A, 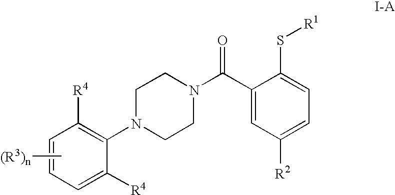
wherein
R¹ is lower alkyl or lower alkyl substituted by halogen;
R² is —S(O)₂-lower alkyl, —S(O)₂NH-lower alkyl, NO₂ or CN;
R³ is halogen, CN, lower alkyl, lower alkyl substituted by halogen, NO₂, —C(O)-lower alkyl or S(O)₂-lower alkyl;
R⁴ is hydrogen or halogen; and n is 0, 1 or 2;
or a pharmaceutically acceptable acid addition salt thereof.

3. A compound of claim 2 wherein R² is —S(O)₂-lower alkyl.

4. A compound of claim 3, wherein R¹ is lower alkyl.

5. A compound of claim 3, wherein R¹ is lower alkyl substituted by halogen.

6. A compound of claim 2, wherein R² is —S(O)₂NH-lower alkyl.

7. A compound of claim 1 having formula I-B 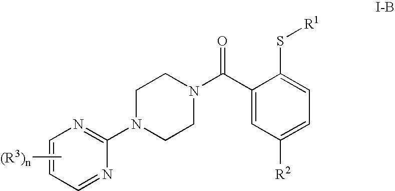
wherein
R¹ is lower alkyl or lower alkyl substituted by halogen;
R² is —S(O)₂-lower alkyl, —S(O)₂NH-lower alkyl, NO₂ or CN;
R³ is halogen, CN, lower alkyl, lower alkyl substituted by halogen, NO₂, —C(O)-lower alkyl or S(O)₂-lower alkyl; and
n is 0, 1 or 2;
or a pharmaceutically acceptable acid addition salt thereof.

8. A compound of claim 7 wherein R² is —S(O)₂-lower alkyl.

9. A compound of claim 8, wherein R¹ is lower alkyl.

10. A compound of claim 8, wherein R¹ is lower alkyl substituted by halogen.

11. A compound of claim 7, wherein R² is —S(O)₂NH-lower alkyl.

12. A compound of claim 1 having formula I-C 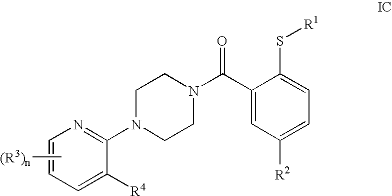
wherein
R¹ is lower alkyl or lower alkyl substituted by halogen;
R² is —S(O)₂-lower alkyl, —S(O)₂NH-lower alkyl, NO₂ or CN;
R³ is halogen, CN, lower alkyl, lower alkyl substituted by halogen, NO₂, —C(O)-lower alkyl or S(O)₂-lower alkyl;
R⁴ is hydrogen or halogen; and
n is 0, 1 or 2;
or a pharmaceutically acceptable acid addition salt thereof.

13. A compound of claim 12 wherein R² is —S(O)₂-lower alkyl.

14. A compound of claim 13, wherein R¹ is lower alkyl.

15. A compound of claim 13, wherein R¹ is lower alkyl substituted by halogen.

16. A compound of claim 12, wherein R² is —S(O)₂NH-lower alkyl.

17. A compound of claim 1, wherein R¹ is lower alkyl.

18. A compound of claim 17, selected from the group consisting of
[4-(3-fluoro-5-trifluoromethyl-pyridin-2-yl)-piperazin-1-yl]-(2-isopropylsulfanyl-5-methanesulfonyl-phenyl)-methanone,
[4-(2-fluoro-4-methanesulfonyl-phenyl)-piperazin-1-yl]-(2-isopropylsulfanyl-5-methanesulfonyl-phenyl)-methanone,
(2-ethylsulfanyl-5-methanesulfonyl-phenyl)-[4-(3-fluoro-5-trifluoromethyl-pyridin-2-yl)-piperazin-1-yl]-methanone, and
[4-(2-fluoro-4-methanesulfonyl-phenyl)-piperazin-1-yl]-(2-isobutylsulfanyl-5-methanesulfonyl-phenyl)-methanone.

19. A compound of claim 17, selected from the group consisting of
1-{3-fluoro-4-[4-(2-isopropylsulfanyl-5-methanesulfonyl-benzoyl)-piperazin-1-yl]-phenyl}-ethanone,
[4-(2-chloro-4-nitro-phenyl)-piperazin-1-yl]-(2-isopropylsulfanyl-5-methanesulfonyl-phenyl)-methanone,
4-isopropylsulfanyl-N-methyl-3-[4-(4-trifluoromethyl-phenyl)-piperazine-1-carbonyl]-benzenesulfonamide and
(2-isopropylsulfanyl-5-nitro-phenyl)-[4-(4-trifluoromethyl-phenyl)-piperazin-1-yl]-methanone.

20. A compound of claim 1, wherein R¹ is lower alkyl substituted by halogen.

21. A compound of claim 20, selected from the group consisting of
[4-(3-fluoro-5-trifluoromethyl-pyridin-2-yl)-piperazin-1-yl]-[5-methanesulfonyl-2-(2,2,2-trifluoro-ethylsulfanyl)-phenyl]-methanone and
[4-(2-fluoro-4-methanesulfonyl-phenyl)-piperazin-1-yl]-[5-methanesulfonyl-2-(2,2,2-trifluoro-ethylsulfanyl)-phenyl]-methanone.

22. A compound of claim 1 wherein R² is —S(O)₂-lower alkyl.

23. A compound of claim 22, wherein R¹ is lower alkyl.

24. A compound of claim 22, wherein R¹ is lower alkyl substituted by halogen.

25. A pharmaceutical composition comprising a compound of formula 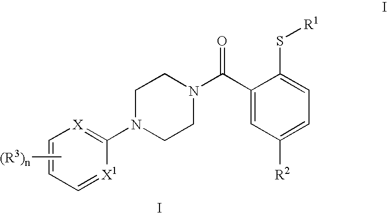
wherein
R¹ is lower alkyl or lower alkyl substituted by halogen;
R² is —S(O)₂-lower alkyl, —S(O)₂NH-lower alkyl, NO₂ or CN;
R³ is halogen, CN, lower alkyl, lower alkyl substituted by halogen, NO₂, —C(O)-lower alkyl or S(O)₂-lower alkyl;
X and X¹ are each independently CR⁴ or N;
R⁴ is hydrogen or halogen; and
n is 0, 1 or 2;
or a pharmaceutically acceptable acid addition salt thereof and a pharmaceutically acceptable carrier.
